(19) United States
(12) Patent Application Publication
(10) Pub. No.: US 2025/0262387 A1
(43) Pub. Date: Aug. 21, 2025

(54) A Label Assembly of a Medicament Delivery Device

(71) Applicant: SHL Medical AG, Zug (CH)

(72) Inventors: Daniel Säll, Nacka Strand (SE); Rasmus Renstad, Nacka Strand (SE); Mattias Myrman, Nacka Strand (SE); Ola Hallström, Nacka Strand (SE)

(21) Appl. No.: 18/857,655

(22) PCT Filed: Apr. 5, 2023
(86) PCT No.: PCT/EP2023/058985

§ 371 Date: Oct. 17, 2024

(30) Foreign Application Priority Data
Apr. 19, 2022 (EP) 22168831.0

(51) Int. Cl. A61M 5/32

(52) U.S. Cl. CPC A61M 5/3202; A61M 2205/3368; A61M 2205/50; A61M 2205/8206

(57) ABSTRACT

The present disclosure provides a label assembly of a medicament delivery device, the label assembly comprising a first label section, a second label section, and a switch. The second label section is releasably connected to the first label section. The switch is formed by a switch part on the first label section and a counter switch part on the second label section. The switch part is connected to the counter switch part when the second label section is connected to the first label section, and the switch part is disconnected to the counter switch part when the second label section are spaced apart from the first label section.

### CROSS-REFERENCE TO RELATED APPLICATIONS

The present application is a U.S. National Phase Application pursuant to 35 U.S.C. § 371 of International Application No. PCT/EP2023/058985 filed Apr. 5, 2023, which claims priority to EP patent application Ser. No. 22/168,831.0 filed Apr. 19, 2022. The entire disclosure contents of these applications are herewith incorporated by reference into the present application.

### TECHNICAL FIELD

The present disclosure generally relates to a label assembly of a medicament delivery device, and especially to a label assembly comprising a switch.

### BACKGROUND

Portable medicament delivery devices such as auto-injectors, inhalers and on-body devices are generally known for the self-administration of a medicament by patients without formal medical training or for emergency use. For example, patients suffering from diabetes or people undertaking an artificial fertilization procedure may require repeated injections of insulin or a hormone. Other patients may require regular injections of other types of medicaments, such as a growth hormone.

To obtain this information from the medicament delivery device, the medicament delivery device can be arranged with several sensors for monitoring and registering e.g., a dose delivery sequence. As medicament delivery devices usually will be stored for a period before being used by users, e.g., a couple of months or a year, it is important to switch all the sensors off during this period. Therefore, there is a demand to make sure all sensors will be switched on right before use of a medicament delivery device.

### SUMMARY

The invention is defined by the appended claims, to which reference should now be made.

In the following description, the wording smart devices will be used. In this context, smart devices may include electronic devices that are provided with processors that are capable of running computer programs as well as storage space to store programs as well as data retrieved from different external sources. It is further to be understood that smart devices are provided with communication systems that are capable of communicating with data networks to access different databases. It is to be understood that databases may be accessed via the internet, so-called cloud services, and/or databases that are connected directly to and accessed via local area networks. It is further to be understood that the smart devices in this context comprise some sort of human-machine interface for two-way communication. The human-machine interface may comprise displays, keyboards, microphones, loudspeakers, and I/O-ports for connection of peripherals. Further, the smart devices may be provided with antennas for wireless communication with the networks. Also, the smart devices may be arranged with receiving and transmitting mechanisms capable of communicating with RFID tags and/or NFC tags as well as programs capable of establishing and handling the communication with the RFID tags and/or NFC tags.

Further, in the following description, the wording medicament delivery device will be used. In this context, medicament delivery devices may include several devices capable of delivering certain doses of medicament to a user, such as e.g. injection devices with or without injection needles, inhalers of all kinds, such as powder, aerosol driven, gas, nebulizers having mouth or nasal pieces, or dispensers for medicament in tablet form. The medicament delivery devices may be either disposable or re-usable and may be provided with medicament containers suitably arranged for specific drugs in specific forms.

There is hence provided a label assembly of a medicament delivery device, the label assembly comprising: a first label section, a second label section, and a switch. The second label section is releasably connected to the first label section. The first label section comprises a transmitter, a memory, a power source and a processor. The transmitter, the memory, the power source and the processor are operably connected. The switch is formed by a switch part on the first label section and a counter switch part on the second label section. The switch part is connected to the counter switch part when the second label section is connected to the first label section. The switch part is disconnected from the counter switch part when the second label section and the first label section are spaced apart. The switch part of the first label section is connected to the processor, and the processor is deactivated when the switch part is connected to the counter switch part.

Preferably, according to one embodiment, the power source is a printed battery.

Preferably, according to one embodiment, the power source is an RF antenna.

Preferably, according to one embodiment, the switch part of the first label section comprises a magnetic reed switch. The counter switch part of the second label section comprises a magnet. The magnetic reed switch is open when the magnet of the counter switch part is connected to the magnetic reed switch of the switch part.

Preferably, according to one embodiment, the switch part of the first label section is positioned between the power source and the processor.

Alternatively, according to one embodiment, the first label section comprises a transparent portion. The switch part of the first label section comprises a photo-detector arranged under the transparent portion of the first label section. The photo-detector of the switch part is electrically connected to the processor and is configured to activate the processor when light is detected. The counter switch part of the second label section comprises a flexible photo-blocking film covering the photo-detector of the switch part when the switch part is connected to the counter switch part.

Alternatively, according to one embodiment, the switch part of the first label section comprises two magnetic conductive pads connected between the printed battery and the processor; wherein the two magnetic conductive pads are configured to magnetically attract one another; and wherein the counter switch part of the second label section comprises a flexible insulating film positioned between the two magnetic conductive pads when the switch part is connected to the counter switch part.

Preferably, according to one embodiment, the first label section comprises a base layer and a flexible film layer movable relative to the base layer. One of the two magnetic conductive pads is arranged on the base layer and the other one of the two magnetic conductive pads is arranged on the flexible film layer.

Alternatively, according to one embodiment, the switch part of the first label section comprises a printed coil. The printed coil of the switch part is electrically connected to the processor. The counter switch part of the second label section comprises a flexible magnetic strip arranged within the printed coil of the switch part of the first label section when the switch part is connected to the counter switch part. The processor is configured to be activated when a pulse on the coil is detected.

Preferably, according to one embodiment, the switch part of the first label section comprises a flexible insulating tube, and the coil is printed on the tube.

Preferably, according to one embodiment, the first label section comprises a temperature sensor connected to the processor.

Alternatively, according to one embodiment, the temperature sensor is a thermoelectric sensor.

Preferably, according to one embodiment, the temperature sensor is arranged between the power source and the processor. The temperature sensor comprises a metallic strip; and wherein the metallic strip deforms or expands to connect the power source to the processor when a predetermined temperature of the first label section is met.

Preferably, according to one embodiment, the metallic strip is a bimetallic strip.

Preferably, according to one embodiment, the switch part and the metallic strip are connected in series.

Preferably, according to one embodiment, the label assembly comprises a tearing interface arranged between the first label section and the second label section so that the connection between the first label section and the second label section is configured to be broken via the tearing interface.

Preferably, according to one embodiment, the tearing interface is a torn line or a tear strip.

Preferably, according to one embodiment, the label assembly is used with a medicament delivery device.

Preferably, according to one embodiment, the medicament delivery device comprises a housing configured to accommodate a medicament container.

Preferably, according to one embodiment, the first label part is attached on an outer surface of the housing.

Preferably, according to one embodiment, the medicament delivery device comprises a cap attached to the housing and configured to be removed from the housing before use.

Preferably, according to one embodiment, the second label part is attached to an outer surface of the cap.

Preferably, according to one embodiment, the tearing interface is aligned with an interface formed between the housing and the cap.

Preferably, according to one embodiment, the medicament delivery device is an injector, an inhaler, a nasal spray, or a medical sprayer.

Preferably, according to one embodiment, the injector is an autoinjector, an infusion pump, a safety syringe or an insulin pen.

Preferably, according to one embodiment, the autoinjector is a hand-hold autoinjector or an on-body autoinjector.

Preferably, according to one embodiment, the cap is configured to cover a needle or a nozzle before use.

Preferably, according to one embodiment, the cap is configured to cover an injection needle before use.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 shows a scenario of using a medicament delivery device with the subject matter of the invention and a smart device.

FIG. 2 schematically shows a medicament delivery device with the subject matter of the invention.

FIGS. 3-7 show block diagrams of different embodiments of the invention.

FIGS. 8-9 schematically show a medicament delivery device different embodiment of the invention.

### DETAILED DESCRIPTION

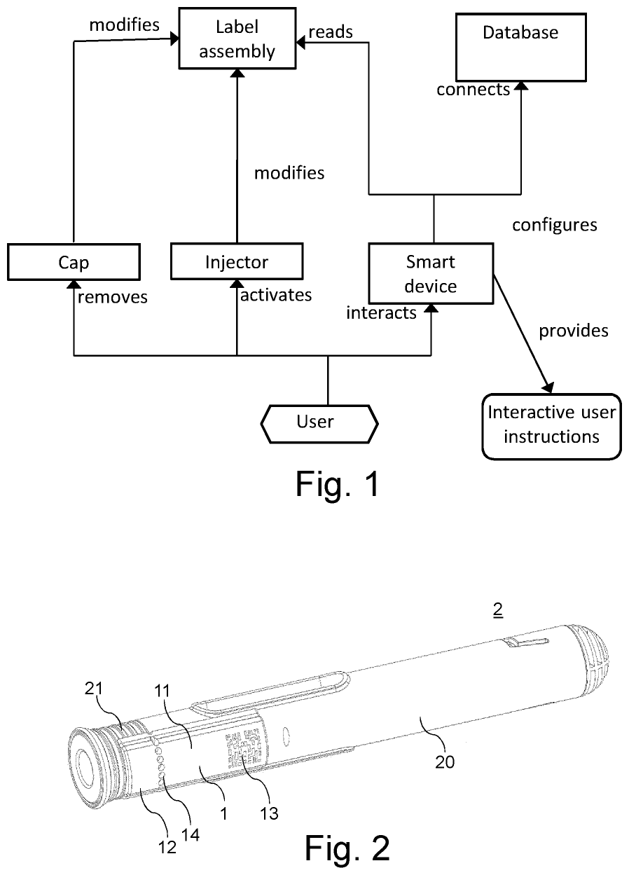

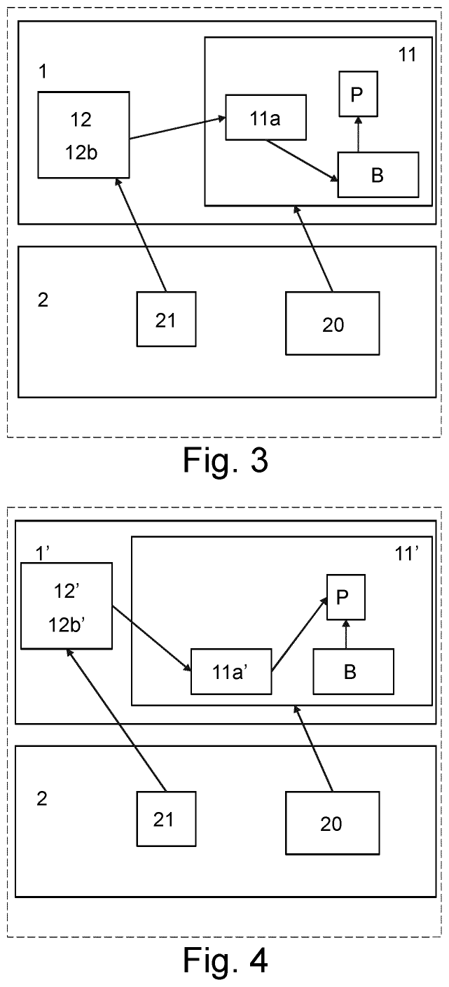

FIGS. 1-9 illustrate label assemblies 1, 1′, 1″, 1′″, 1″″ of the invention. The label assembly 1, 1′, 1″, 1′″, 1″″ is configured to be attached to a medicament delivery device. The label assembly 1, 1′, 1″, 1′″, 1″″ comprises a first label section 11, 11′, 11″, 11′″, 11″″, a second label section 12, 12′, 12″, 12′″, 12″″, and a switch. The second label section 12, 12′, 12″, 12′″, 12″″ is releasably connected to the first label section 11, 11′, 11″, 11′″, 11″″. In a preferred example, the first label section 11 and the second label section 12 form one label sheet and are configured to be torn apart from a tearing interface arranged between the first label section 11 and the second label section 12, as shown in FIG. 2. Therefore, the connection between the first label section 11 and the second label section 12 is released when the label sheet that is formed by the first label section 11 and the second label section 12 is torn. In one example, the label sheet is adhesive and attached to the medicament delivery device. In particular, the label sheet is attached to the outer surfaces of two components of the medicament delivery device respectively. The two components of the medicament delivery device are configured to be moved apart from one another before the user uses the medicament delivery device. In one example, the tearing interface 14 is a torn line 14, e.g., with a pattern of a plurality of holes positioned in series or a zigzag line. In this example, when the user moves the two components apart, the label sheet can be torn apart into the first label section and the second label section from the torn line 14 and thereby moved apart from the first label section. In another example, the tearing interface is defined by a tear strip. In this example, the first label section and the second label section can be formed as two independent sheets that are attached to two components of the medicament delivery device respectively, and next to one another. An adhesive tape with the tearing strip is attached to the first label section and the second label section with the tearing strip lined up with an interface between the first label section and the second label section. Therefore, the connection between the first label section and the second label section can be released when the tearing strip is torn. In this example, when the user plans to use the medicament delivery device, the user needs to tear the tearing strip and thereby rupture the adhesive tape; then the user can move the two components of the medicament delivery device apart, thereby moving the first label section and the second label section apart. Alternatively, the first label section and the second label section can be attached via the tearing strip. In this example, when the user plans to use the medicament delivery device, the user directly moves the two components of the medicament delivery device apart to tear the tearing strip, thereby moving the first label section and the second label section apart.

The first label section and the second label section are respectively attached to two components of the medicament delivery device that are configured to be moved apart from one another before use of the medicament delivery device, for example, the two components can be a housing and a cap, or a housing and a safety pin/clip. Therefore, the first label section and the second label section will eventually be spaced apart from one another, together with the two components of the medicament delivery device.

In one example where the medicament delivery device 2 comprises a housing 20 for accommodating a medicament and a cap 21 for attaching to the housing and thereby covering a medicament delivery member, one of the first label section 11 and the second label section 12 is attached to the housing 20, and the other one of the first label section 11 and the second label section 12 is attached to the cap 21. In the example, as shown in FIG. 2, the label assembly is used with a medicament delivery device comprising the housing 20 configured to accommodate a medicament container. In a preferred example, the first label part 11 is attached to an outer surface of the housing 20. The medicament delivery device comprises a cap 21 attached to the housing and configured to be removed from the housing before use. In this example, the second label part 12 is attached to an outer surface of the cap 21. In one example where the label assembly comprises the tearing interface 14, the tearing interface 14 is optionally aligned with an interface formed between the housing 20 and the cap 21, as shown in FIG. 2.

In a preferred example, the medicament delivery device is an injector. In this example, the cap is configured to cover an injection needle before use.

In another example where the medicament delivery device comprises a housing for accommodating a medicament container and a safety pin for locking the medicament delivery device from unintentionally triggering, one of the first label section and the second label section is attached to the housing, and the other one of the first label section and the second label section is attached to the cap.

The first label section 11, 11′, 11″, 11′″, 11″″ comprises a transmitter, a memory, a power source B and a processor P. The transmitter, the memory, the power source B and the processor P are operably connected. The processor P can be a thin-film microprocessor or an NFC microcontroller, for example. The memory can be an inkjet print-programmable memory, for example. Alternatively, the first label section comprises an RFID or NFC chip; in this example, the processor P and the memory are in the RFID or NFC chip. The transmitter can be an RFID or NFC antenna, for example. In one example, the power source is a printed battery (also called a ‘thin-film’ battery) so that other electronics can have a power supply when connected to the printed battery electrically. In another example, the power source is an RF antenna so that the other electronics can have a power supply when connected to the printed battery electrically and the first label section is positioned within an area that can receive a predetermined RF signal, for example, when the first label section is adjacent to an RF wireless charging pad or is located within the area covered by RF charging signal.

The switch 11a, 12b; 11a′, 12b′; 11a″, 12b″; 11a′″, 12b″″ is formed by a switch part 11a, 11a′, 11a″, 11a′″ on the first label section 11, 11′, 11″, 11′″, 11″″ and a counter switch part 12b, 12b′, 12b″, 12b′″ on the second label section 12, 12′, 12″, 12′″, 12″″. Therefore, as mentioned above, when the first label section 11, 11′, 11″, 11′″, 11″″ is connected to the second label section 12, 12′, 12″, 12′″, 12″″, e.g., either the first label section and the second label section are formed as one label sheet, or the first label section and the second label section are next to one another, and the switch part 11a, 11a′, 11a″, 11a′″ is connected to the counter switch part 12b, 12b′, 12b″, 12b′″. When the second label section 12, 12′, 12″, 12′″, 12″″ is spaced apart from the first label section 11, 11′, 11″, 11′″, 11″″, the switch part 11a, 11a′, 11a″, 11a′″ is not connected to the counter switch part 12b, 12b′, 12b″, 12b′″. The switch part 11a, 11a′, 11a″, 11a′″ ′ of the first label section 11, 11′, 11″, 11′″, 11″″ is connected to the processor; and the processor is deactivated when the switch part 11a, 11a′, 11a″, 11a′″ is connected to the counter switch part 12b, 12b′, 12b″, 12b′″.

As the switch 11a, 12b; 11a′, 12b′; 11a″, 12b″; 11a′″, 12b′″ is configured to make sure the processor can be switched on before a use of the medicament delivery device to which the label assembly 1, 1′, 1″, 1′″, 1″″ is attached, as long as the switch part 11a, 11a′, 11a″, 11a′″ and the counter switch part 12b, 12b′, 12b″, 12b′″ are not connected to one another when the first label section 11, 11′, 11″, 11′″, 11″″ and the second label section 12, 12′, 12″, 12′″, 12″″ are spaced apart (as mentioned above, that means the two removably attached components of the medicament delivery device are spaced apart), the processor can be switched on, on time, by the switch 11a 12b; 11a′, 12b′; 11a″, 12b″; 11a′″, 12b′″. Therefore, the exact starting point that the switch part 11a, 11a′, 11a″, 11a′″ and the counter switch part 12b, 12b′, 12b″, 12b′″ are disconnected from one another is dependent on the design. For example, the switch can be designed so that the switch part and the counter switch part are disconnected from one another once the label sheet that is formed by the first label section and the second label section is partially torn, or fully torn but with the first label section and the second label section still arranged next to one another, or the switch part and the counter switch part will only disconnect from one another when the first label section and the second label section are spaced apart from one another.

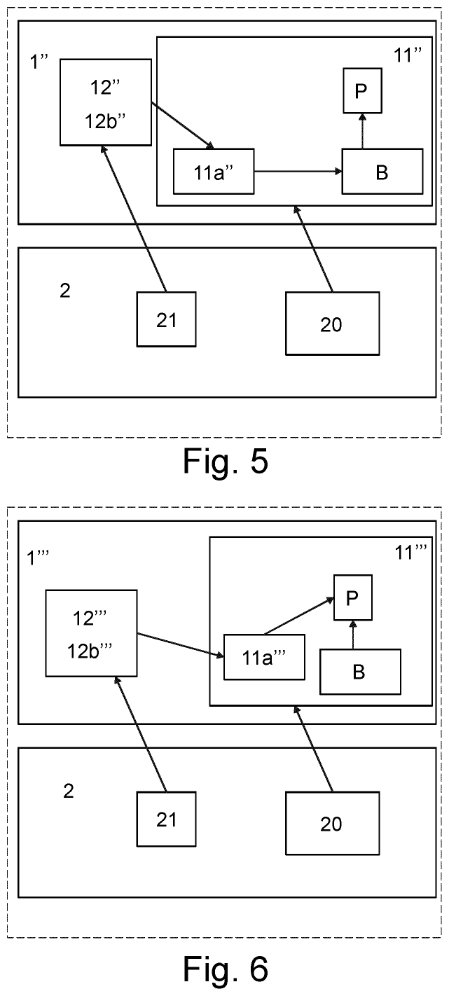

In one example, as shown in FIGS. 3-5 and FIG. 8, the switch 11a, 12b; 11a′, 12b′; 11a″, 12b″ comprises a magnet.

In one example, as shown in FIG. 3, the switch part 11a of the first label section 11 comprises a magnetic reed switch. The counter switch part 12b of the second label section 12 comprises a magnet. The magnetic reed switch is open when the magnet of the counter switch part 12b is connected to the magnetic reed switch of the switch part 11a. In a preferred example, the switch part 11a of the first label section 11 is positioned between the power source B and the processor P so that the processor P can only be powered on by the power source B when the magnetic reed switch is closed. Alternatively, the magnetic reed switch of the switch part 11a only connects to the processor P. In this example, the processor P is configured to be activated when a switching-on signal from the closed magnetic reed switch is received. In this example, the processor can be designed to always stay in the ‘on’ state once the processor has been switched on so that even if there is any change in the status of the switch, e.g., the magnetic reed switch is reopened when the user reattaches the cap to the housing, the processor P will not be turned off.

In another example, as shown in FIG. 4 and FIG. 9, the switch part 11a′ of the first label section 11′ comprises a coil 11a′. The coil of the switch part 11a′ is electrically connected to the processor P. The counter switch part 12b′ of the second label section 12′ comprises a magnetic strip 12b′ arranged within the coil 11a′ of the switch part 11a′ of the first label section 11′ when the switch part 11a′ is connected to the counter switch part 12b′. The movement of the magnetic strip 12b′ induces current on the coil, in this example, and the processor P is configured to be activated when an electric pulse on the printed coil 12b′ is detected. In one example, the switch part 11a′ of the first label section 11′ comprises a flexible insulating tube. In a preferred example, the coil is printed on the flexible insulating tube. In another preferred example, the tube is made of a thin plastic film. In another preferred example, the magnetic strip is flexible. In one example, as shown in FIG. 9, the first label section 11′ comprises a base conductive layer 11c′ and a conductive film layer 11d′; the base conductive layer 11c′ and the conductive film layer 11d′ are connected to the processor P. The printed coil 11a′ is positioned between the base conductive layer 11c′ and the conductive film layer 11d′ so that the induced current can be detected by the process P.

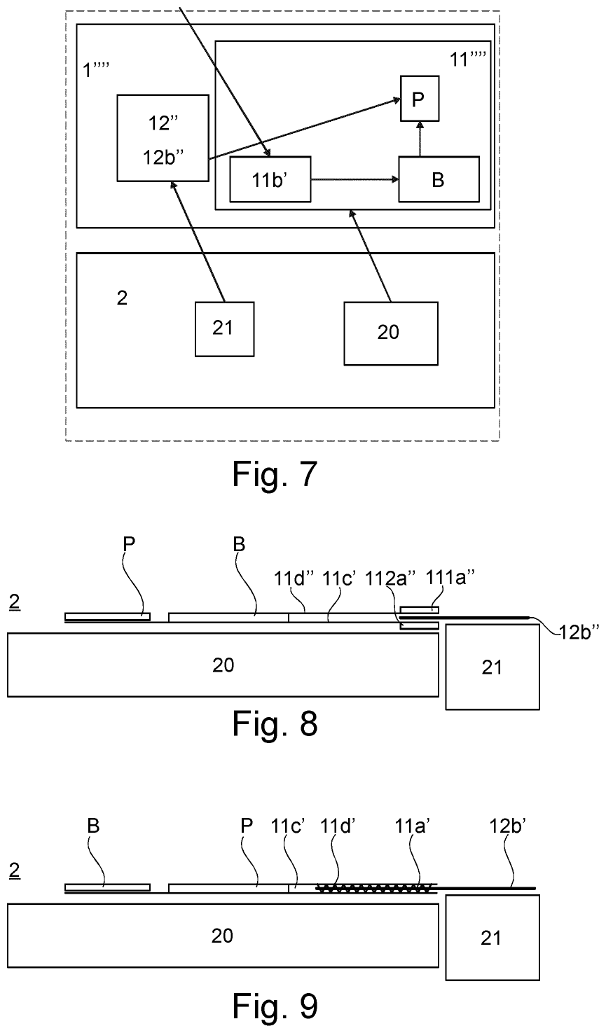

In another example, as shown in FIG. 5 and FIG. 8, the switch part 11a″ of the first label section 11″ comprises two magnetic conductive pads 111a″, 112a″ connected between the printed battery B and the processor P. The two magnetic conductive pads 111a″, 112a″ are configured to magnetically attract one another. The counter switch part 12b″ of the second label section 12′″ comprises a flexible insulating film 12b″ positioned between the two magnetic conductive pads 111a″, 112a″ when the switch part 11a″ is connected to the counter switch part 12b″. Therefore, when the switch part 11a″ is not connected to the counter switch part 12b″, the flexible insulating film 12b″ can move away from the two magnetic conductive pads 111a″, 112a″ and thereby the two magnetic conductive pads 111a″, 112a″ contact one another and activate the processor P. Similarly, as in the above-mentioned example, the switch part 11a″ of the first label section 11″ is positioned between the power source B and the processor P so that the processor P can only be powered on by the power source B when the two magnetic conductive pads 111a″, 112a″ contact one another. Alternatively, the switch part 11a″ only connects to the processor P. In this example, the processor P is configured to be activated when a signal from a closed loop is formed by the connection of the two magnetic conductive pads.

In a preferred example, as shown in FIG. 8, the first label section 11″ comprises the base conductive layer 11c′ and a flexible film layer 11d″. The flexible film layer 11d″ is movable relative to the base conductive layer 11c′. One of the two magnetic conductive pads 112a″ is arranged on the base conductive layer 11c″ and the other one of the two magnetic conductive pads 111a″ is arranged on the flexible film layer 11d″. The flexible film layer 11d″ comprises a conductive material.

In another example, the switch comprises a bubble containing conductive liquid arranged between the base conductive layer of the switch part and the flexible conductive layer of the switch part. The bubble is made of isolating material. Outside the bubble is a porous absorbing material that is isolating when dry but conductive when it has absorbed conductive liquid. In this example, the counter switch part comprises a bubble break tab. Therefore, when the switch part and the counter switch part are disconnected, the bubble break tab makes the bubble burst. The liquid is absorbed by the porous material so that it becomes conductive and the electric connection between the base conductive layer and the flexible conductive layer is established. In this example, the base conductive layer and the flexible conductive layer can be arranged to only connect to the processor or connect to both the processor and the power source.

In another example, as shown in FIG. 6, the first label section 11′″ comprises a transparent portion. The switch part 11a′″ of the first label section 11′″ comprises a photo-detector 11a′″ arranged under the transparent portion of the first label section 11′″. The photo-detector 11a′″ of the switch part 11a′″ is electrically connected to the processor P and is configured to activate the processor P when light is detected. The counter switch part 12b′″ of the second label section 12′″ comprises a flexible photo-blocking film 12b′″, e.g., a dark color film, covering the photo-detector 11a′″ of the switch part 11a′″ when the switch part 11a′″ is connected to the counter switch part 12b′″. Therefore, when the switch part is disconnected to the counter switch part, the photo-blocking film 12b′″ can move out from the transparent portion of the first label section 11′″ and thereby the photo-detector 11a′″ detects the ambient light and activates the processor. Similarly, as in the above-mentioned example, the processor can be designed to always stay in the ‘on’ state once the processor has been switched on so that even if there is any change in the status of the switch, e.g., the transparent portion is blocked by the user during use of the medicament delivery device, the processor P will not be turned off.

In another example, the first label section 11″″ comprises a temperature sensor 11b′ connected to the processor P, as shown in FIG. 7. The temperature sensor is configured to detect the temperature of the first label section 11″″. As the first label section 11″″ is configured to be attached to a medicament delivery device, the temperature of the first label section 11″″ will be similar to the temperature of the medicament delivery device to which the first label section 11″″ is attached.

Some medicaments need to be used at room temperature, e.g., 20-25 degrees Celsius, but need to be stored at a relatively low temperature, e.g., 4-8 degrees Celsius in a refrigerator. The temperature sensor of the first label section 11″″ can be used either as a further switch to switch on the processor P when the medicament delivery device reaches a temperature that is suitable to be used or to record the temperature information. In the latter example, the temperature sensor can be used together with any other switch 11a, 12b; 11a′, 12b′; 11a″, 12b″; 11a′″, 12b′″ as mentioned above.

In one example, the temperature sensor is a thermoelectric sensor. In another example, the temperature sensor comprises a metallic strip, and the metallic strip deforms or expands when a predetermined temperature of the first label section 11a″″ is reached. The metallic strip can be arranged to only contact the processor P or to contact both the processor P and the power source B. In a preferred example, the metallic strip is arranged between the power source B and the processor P. In this example, the metallic strip deforms or expands to connect the power source B to the processor P when a predetermined temperature of the first label section 11″″ is met. In a preferred example, the metallic strip is a bimetallic strip.

In one example where the metallic strip is arranged between the power source B and the processor P. In this example the label assembly can also comprise the switch 11a, 12b comprising the switch part 11a, wherein the switch part 11a comprises the magnetic reed switch arranged between the power source B and the processor P, as mentioned above, or the switch 11a″, 12b″ comprising the switch part 11a″, wherein the switch part 11a″ comprises the two conductive pads connected to the processor P and the power source B, as mentioned above. In this example, the switch part 11a, 11a″ and the metallic strip 11b′ are connected in series.

The label assembly can be arranged with more sensors or indicators electrically connected to the processor, dependent on the design. For example, the label assembly can comprise a thermochromic material as a temperature indicator for the user. Furthermore, the label assembly can be printed with information 13 in a format of textures, figures, barcodes and/or a QR code. The label assembly can be made of label sheets that are thin, e.g., 0.3-1 mm, and flexible. Alternatively, the label assembly can be made of label tags.

One example of using the label assembly is shown in FIG. 1. In this example, when the user uses an injector, the user will remove the cap first. The removal of the cap will switch on the processor. The processor can be used to control all other sensors to detect the action of the injection device or record the usage of the injector. The record can be sent to a remote device directly or can be stored in the memory and read by a smart device.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are described in the clauses below.
1. A label assembly of a medicament delivery device, the label assembly comprising: a first label section, a second label section, and a switch;
   wherein the second label section is releasably connected to the first label section;
   wherein the first label section comprises a transmitter, a memory, a power source and a processor; wherein the transmitter, the memory, the power source and the processor are operably connected;
   wherein the switch is formed by a switch part on the first label section and a counter switch part on the second label section;
   wherein the switch part is connected to the counter switch part when the second label section is connected to the first label section; wherein the switch part is not connected to the counter switch part when the second label section is spaced apart from the first label section; and
   wherein the switch part of the first label section is connected to the processor; and wherein the processor is deactivated when the switch part is connected to the counter switch part.
2. The label assembly according to clause 1, wherein the power source is a printed battery.
3. The label assembly according to clause 1, wherein the power source is an RF antenna.
4. The label assembly according to any of the preceding clauses, wherein the switch comprises a magnet.
5. The label assembly according to clause 4, wherein the switch part of the first label section comprises a magnetic reed switch; wherein the counter switch part of the second label section comprises a magnet; and wherein the magnetic reed switch is open when the magnet of the counter switch part is connected to the magnetic reed switch of the switch part.
6. The label assembly according to clause 5, wherein the switch part of the first label section is positioned between the power source and the processor.
7. The label assembly according to clause 4, wherein the first label section comprises two magnetic conductive pads connected between the printed battery and the processor; wherein the two magnetic conductive pads are configured to magnetically attract one another; and wherein the counter switch part of the second label section comprises a flexible insulating film positioned between the two magnetic conductive pads when the switch part is connected to the counter switch part.
8. The label assembly according to clause 7, wherein the first label section comprises a base conductive layer and a flexible conductive film layer movable relative to the base conductive layer; wherein one of the two magnetic conductive pads is arranged on the base conductive layer and the other one of the two magnetic conductive pads is arranged on the flexible conductive film layer.
9. The label assembly according to clause 7 or 8, wherein one of the base conductive layer and the flexible conductive film layer is connected to the processor and the other one of the base conductive layer and the flexible conductive film layer is connected to the power source.
10. The label assembly according to any clause 4, wherein the switch part of the first label section comprises a coil; wherein the coil of the switch part is electrically connected to the processor; wherein the counter switch part of the second label section comprises a magnetic strip arranged within the coil of the switch part of the first label section when the switch part is connected to the counter switch part; and wherein the processor is configured to be activated when a pulse on the coil is detected.
11. The label assembly according to clause 10, wherein the switch part of the first label section comprises a flexible insulating tube; and wherein the coil is printed on the tube.
12. The label assembly according to any of clauses 1-3, wherein the first label section comprises a transparent portion; wherein the switch part of the first label section comprises a photo-detector arranged under the transparent portion of the first label section; wherein the photo-detector of the switch part is electrically connected to the processor and is configured to activate the processor when light is detected; and wherein the counter switch part of the second label section comprises a flexible photo-blocking film covering the photo-detector of the switch part when the switch part is connected to the counter switch part.
13. The label assembly according to any of the preceding clauses, wherein the first label section comprises a temperature sensor connected to the processor.
14. The label assembly according to clause 13, wherein the temperature sensor is a thermoelectric sensor.
15. The label assembly according to clause 13, wherein the temperature sensor is arranged between the power source and the processor; wherein the temperature sensor comprises a metallic strip; and wherein the metallic strip deforms or expands to connect the power source to the processor when a predetermined temperature of the first label section is met.
16. The label assembly according to clause 15, wherein the metallic strip is a bimetallic strip.
17. The label assembly according to clause 15 or 16, when dependent on clause 6 or 9, wherein the switch part and the metallic strip are connected in series.
18. The label assembly according to any of the preceding clauses, wherein the label assembly comprises a tearing interface arranged between the first label section and the second label section so that the connection between the first label section and the second label section is configured to be broken via the tearing interface.
19. The label assembly according to clause 18, wherein the tearing interface is a torn line or a tear strip.
20. A medicament delivery device comprising the label assembly according to any of the preceding clauses.
21. The medicament delivery device according to clause 20, comprising a housing configured to accommodate a medicament container; and wherein the first label part is attached on an outer surface of the housing; wherein the medicament delivery device comprises a cap attached to the housing and configured to be removed from the housing before use; wherein the second label part is attached to an outer surface of the cap.
22. The medicament delivery device according to clause 21, wherein the medicament delivery device is an injector; wherein the cap is configured to cover an injection needle before use.
23. The medicament delivery device according to any of clauses 20-22 when dependent on clause 18 or 19, wherein the tearing interface is aligned with an interface formed between the housing and the cap.

1-15. (canceled)

16. A label assembly of a medicament delivery device, the label assembly comprising:
a first label section;
a second label section; and
a switch,
wherein the second label section is releasably connected to the first label section,
wherein the first label section comprises a transmitter, a memory, a power source and a processor,
wherein the transmitter, the memory, the power source and the processor are operably connected,
wherein the switch is formed by a switch on the first label section and a counter switch part on the second label section,
wherein the switch part is connected to the counter switch part when the second label section is connected to the first label section,
wherein the switch part is not connected to the counter switch part when the second label section is spaced apart from the first label section,
wherein the switch part of the first label section is connected to the processor, and
wherein the processor is deactivated when the switch part is connected to the counter switch part.

17. The label assembly according to claim 16, wherein the switch comprises a magnet.

18. The label assembly according to claim 17, wherein the switch part of the first label section comprises a magnetic reed switch, wherein the counter switch part of the second label section comprises a magnet, and wherein the magnetic reed switch is open when the magnet of the counter switch part is connected to the magnetic reed switch of the switch part.

19. The label assembly according to claim 18, wherein the switch part of the first label section is positioned between the power source and the processor.

20. The label assembly according to claim 17, wherein the first label section comprises two magnetic conductive pads connected between the printed battery and the processor, wherein the two magnetic conductive pads are configured to magnetically attract one another, and wherein the counter switch part of the second label section comprises a flexible insulating film positioned between the two magnetic conductive pads when the switch part is connected to the counter switch part.

21. The label assembly according to claim 20, wherein the first label section comprises a base conductive layer and a flexible conductive film layer movable relative to the base conductive layer, wherein one of the two magnetic conductive pads is arranged on the base conductive layer and the other one of the two magnetic conductive pads is arranged on the flexible conductive film layer.

22. The label assembly according to claim 20, wherein one of the base conductive layer and the flexible conductive film layer is connected to the processor and the other one of the base conductive layer and the flexible conductive film layer is connected to the power source.

23. The label assembly according to any claim 17, wherein the switch part of the first label section comprises a coil, wherein the coil of the switch part is electrically connected to the processor, wherein the counter switch part of the second label section comprises a magnetic strip arranged within the coil of the switch part of the first label section when the switch part is connected to the counter switch part, and wherein the processor is configured to be activated when a pulse on the coil is detected.

24. The label assembly according to claim 23, wherein the switch part of the first label section comprises a flexible insulating tube; and wherein the coil is printed on the tube.

25. The label assembly according to any of claim 16, wherein the first label section comprises a transparent portion, wherein the switch part of the first label section comprises a photo-detector arranged under the transparent portion of the first label section, wherein the photo-detector of the switch part is electrically connected to the processor and is configured to activate the processor when light is detected, and wherein the counter switch part of the second label section comprises a flexible photo-blocking film covering the photo-detector of the switch part when the switch part is connected to the counter switch part.

26. The label assembly according to claim 16, wherein the first label section comprises a temperature sensor connected to the processor.

27. The label assembly according to claim 26, wherein the temperature sensor is arranged between the power source and the processor, wherein the temperature sensor comprises a metallic strip, and wherein the metallic strip deforms or expands to connect the power source to the processor when a predetermined temperature of the first label section is met.

28. The label assembly according to claim 27, wherein the metallic strip is a bimetallic strip.

29. A medicament delivery device comprising the label assembly according to claim 16, wherein the medicament delivery device comprises a housing configured to accommodate a medicament container, wherein the first label part is attached on an outer surface of the housing, wherein the medicament delivery device comprises a cap attached to the housing and configured to be removed from the housing before use, and wherein the second label part is attached to an outer surface of the cap.

30. The medicament delivery device according to claim 29, wherein the medicament delivery device is an injector; wherein the cap is configured to cover an injection needle before use.
